(19) United States
(12) Patent Application Publication
(10) Pub. No.: US 2009/0036576 A1
(43) Pub. Date: Feb. 5, 2009

(54) Cyanophycin Production From Nitrogen-Containing Chemicals Obtained From Biomass

(76) Inventors: Yasser Abdel Kader Elbahloul, Munster (DE); Elinor Lindsey Scott, Amersfoort (NL); Andreas Mooibroek, Renkum (NL); Johan Pieter Marinus Sanders, Groningen (NL); Martin Obst, Munster (DE); Alexander Steinbuchel, Altenberge (DE)

Correspondence Address: MORGAN LEWIS & BOCKIUS LLP, 1111 PENNSYLVANIA AVENUE NW, WASHINGTON, DC, 20004, US

(21) Appl. No.: 11/817,741

(22) PCT Filed: Mar. 6, 2006
(86) PCT No.: PCT/NL2006/050047

§ 371 Date: Sep. 3, 2008

(30) Foreign Application Priority Data
Mar. 4, 2005 (EP) 05101713.5

(51) Int. Cl. C08L 89/00; C12P 21/02; C12P 21/06; C12P 13/20; C12P 13/10; C12P 13/00; C12P 7/46; C12P 7/04; C07C 229/24

(52) U.S. Cl. 524/17; 435/71.2; 435/69.1; 435/68.1; 435/109; 435/114; 435/128; 435/145; 435/157; 562/571

(57) ABSTRACT

The present invention relates to fermentation processes for the production of cyanophycin in a microorganism whereby a plant-derived nitrogen source is converted by the microorganism into cyanophycin. The plant-derived nitrogen source preferably is a process stream being obtained in the processing of agricultural crops such as e.g., a by-product in the processing of starch from agricultural crops like corn, potato or cassave. The invention further relates to processes for the conversion of cyanophycin into a variety of compounds including e.g., ornithine, 1,4-butanediamine, n-alkyl amino alcohols, acrylonitrile, as well as cyanophycin derived functionalised poly(aspartic acid)s wherein the arginine residues have been functionalised to ornithine, (N-L-arginino)succinate, N-phospho-L-arginine or agmantine and the lysine residues have been functionalised to N6-hydroxy-L-lysine, 2,5-diaminohexanoate, N6-(L-1,3-dicarboxypropyl), pentanediamine, 5-aminopentanamide or N6-acetyl-L-lysine. These functionalised groups can be further subjected to subsequent chemical and/or enzymatic modifications.

### FIELD OF THE INVENTION

The present invention relates to fermentation processes for the production of cyanophycin in a microorganism whereby a plant-derived nitrogen source is converted by the microorganism into cyanophycin. The invention further relates to processes for the conversion of cyanophycin into a variety of compounds, preferably nitrogen-containing compounds.

### BACKGROUND OF THE INVENTION

Cyanophycin (also referred to as CGP: Cyanophycin Granule Polypeptide) was discovered in 1887 by Borzi during microscopic studies of cyanobacteria (Borzi, 1887) and was later found in all groups of Cyanobacteria (Oppermann-Sanio et al., 2003). The CGP molecule structure is related to that of poly(aspartic acid)s, but, unlike synthetic poly-aspartic acid, it is a comb-like polymer with α-amino-α-carboxy-linked L-aspartic acid residues representing the poly(α-L-aspartic acid) backbone and L-arginine residues bound to the β-carboxylic groups of aspartic acids. Cyanophycin isolated from Cyanobacteria is highly polydisperse and shows a molecular weight range of 25-100 kDa as estimated by SDS-PAGE corresponding to a polymerization degree of 90-400 (Simon, 1971; Simon and Weathers, 1973; Simon and Weathers, 1976). Cyanophycin is a transiently accumulated storage compound which is synthesized under conditions of low temperature or low light intensity. Its accumulation can be artificially enhanced by the addition of chloramphenicol as an inhibitor of ribosomal protein biosynthesis (Simon, 1973). Cyanophycin plays an important role in the conservation of nitrogen, carbon, and energy and, as the resistance toward chloramphenicol indicated, is non-ribosomally synthesized by cyanophycin synthetases (CphA). Cyanophycin is accumulated in the cytoplasm of cyanobacteria as membraneless granules (Allen and Weathers, 1980) in the early stationary growth phase (Mackerras et al., 1990; Liotenberg et al., 1996). When growth is resumed, for example due to a change in culture conditions, cyanophycin is reutilized by the cells Mackerras et al. (1990). Krehenbrink et al. and Ziegler et al. showed that cyanophycin occurs even in heterotrophic bacteria like Acinetobacter sp. and Desulfitobacterium hafniense and therefore confirmed the wide distribution of this biopolymer and its function in nature as a general storage compound (Krehenbrink et al., 2002; Ziegler et al., 2002).

The biosynthesis of cyanophycin was extensively studied in the 1970s by Simon and co-workers (Simon, 1971; Simon and Weathers, 1973; Simon and Weathers, 1976; Simon, 1973; Simon, 1976), which led to the identification of cyanophycin-synthetases and the genes encoding the enzymes (cphA) in various organisms (Ziegler et al., 1998; Aboulmagd et al., 2000; Berg et al., 2000; Hai et al., 2002). Subsequently the enzymes involved in the degradation of cyanophycin by intracellular CGPases of cyanobacteria and their genes (cphB) were identified. However, cyanophycin is highly resistant against hydrolytic cleavage by proteases such as trypsin, pronase, pepsin, carboxypeptidases B, carboxypeptidase C, and leucin-aminopeptidase (Simon and Weathers, 1976) and cyanophycin is also resistant against arginases (Simon, 1987). Various processes have been described in the art for the production of cyanophycin employing cyanobacterial cells. DE-A 197 09 024 e.g. discloses the extraction and purification of cyanophycin from Aphanocapsa PCC 6308 and Hai et al. (1999) disclose the production of cyanophycin using Synechococcus sp. MA 19.

Several publications disclose the isolation of the cyanophycin synthetase genes, e.g. from Synechocystis PCC 6803 or Anabaena variabilis ATCC 29 413 (DE-A 19813692) allowing for the production of cyanophycin in recombinant bacteria, including bacteria other than cyanobacteria. E.g. production of cyanophycin by recombinant bacteria in 30-500 L scale has been reported (Aboulmagd et al., 2001; Frey et al., 2002), which make cyanophycin now available in larger quantities. WO0212508 relates to thermostable cyanophycin synthetases and a method for the improved production of cyanophycin and/or the secondary products thereof.

The economically feasible production of cyanophycin by fermentation using the constituting amino acids arginine and aspartate are far too expensive when these amino acids are obtained by sugar-based fermentation or enzymatic catalysis, respectively (Leuchtenberger, 1996). On the other hand, the fermentation yield on sugar and ammonia is too low. There is thus still a need in the art for an economically feasible route to the production of cyanophycin for high-value specialty applications of cyanophycin, such as in medical or surgical devices, (food) packaging materials and coatings. However, the invention also provides for cheap industrial bulk production processes starting from Protamylasse™ and other cheap waste streams can be developed for cyanophycin-based amino acids and derived products, such as arginine, polyaspartic acid and ornithine. The invention thus also provides for methods for valorising N-containing waste streams of plant materials.

### DESCRIPTION OF THE INVENTION

To our knowledge there are no examples of nitrogen-containing chemicals that are produced on an economically feasible industrial scale using nitrogen-containing plant materials as the only raw materials, i.e. without the addition of nitrogen sources like ammonia. Large-scale fermentation processes exist for the production of feed- and food-grade amino acids lysine and glutamic acid. However, the fermentative production of these compounds using carbohydrates and ammonia is far too expensive to use them as starting materials for N-containing bulk chemicals, such as diamines and acrylonitrile. While in the case of fermentation processes that start with carbohydrates we anticipate that the raw materials will become cheaper, but this is not the case for the nitrogen-containing chemicals since they make use of ammonia that can only be obtained from the nitrogen fixation process by using a large amounts of fossil resources. Some plants, such as e.g. Legumes, can bind nitrogen from the air. Therefore, the use of nitrogen-containing plant-derived material as nitrogen source in microbial fermentation would allow producing a variety of nitrogen-containing polymers that can be recovered at low cost. These polymers can also serve as the source of building blocks in chemical and feed industries. One such nitrogen-containing polymer is cyanophycin that occurs in certain cyanobacteria, probably as an insoluble storage molecule for nitrogen.

The current inventors have found that cyanophycin unexpectedly appeared very suitable as a starting molecule for the recovery of amino acids from biomass. With its polymeric backbone of aspartic acids, to each unit of which an arginine unit is coupled, cyanophycin contains 5 N-atoms per aspartic acid-arginine monomer of which the polymer is composed. Because of their biocompatibility, their synthesis from renewable resources and chiral functionality, cyanophycins may be employed for many different purposes covering a broad spectrum of medical, pharmaceutical, optical and personal care applications as well as the domains of agriculture and of environmental applications such as coatings and other polymer applications. The biocompatibility and complete biodegradability of cyanophycins makes them ideal candidates for many applications in human life. Because of their low environmental impact, these biopolymers could substitute synthetic polymers with similar characteristics in the fields of biomedicine, agriculture, agrochemistry, personal care, optical applications and pharmacy such as coatings and other polymer applications. An increasing future demand for biopolymers will help to reduce environmental pollution caused by chemosynthetic polymers such as plastics or synthetic, ionic residues containing polymers (ionomers) which are often used for short time applications but show a long residence time in nature. Such polymers are, based on their material properties, in most cases not biodegradable (Aboulmagd et al., 2000) or they are only partially degraded but not completely mineralized after release into the environment. In terms of sustainability and to become more independent of petrochemical resources, bio-based and bio-degradable polymers such as poly(amino acid)s will become more and more attractive alternatives. Along with an increasing number of applications, an increasing demand for environmentally friendly (bio)polymers, and decreasing production costs, the market for biopolymers will most probably expand more rapidly during the next decades. Furthermore, chemical and/or enzymatic modification of cyanophycins or derivatives thereof can yield a variety of polymeric properties. It is known that upon hydrolysis of the arginine-aspartic acid bond polyaspartate is being formed with properties that are very similar to poly(acrylic acid). Because of the structural similarity of cyanophycin and poly(aspartic acid), alternative degradation mechanisms for cyanophycin, initiated by hydrolytic β-cleavage leading to the release of free arginine from cyanophycin, would be desirable for the formation of poly(α-aspartic acid) for which many technical applications are known (see above). Such a process has a high market potential if the production costs are sufficiently low. In addition, the arginine and aspartic acid obtainable from the cyanophycin molecule can serve as building blocks for the synthesis of a variety of chemicals that contain nitrogen. Arginine in the presence of arginase has been described to form urea and ornithine. The ornithine may subsequently be treated with ornithine decarboxylase to form 1,4-butanediamine, a monomer used in the synthesis of nylon-4,6 and CO₂.

In a first aspect the present invention therefore relates to a process for producing cyanophycin. The process of the invention preferably comprises the conversion of nitrogen and optionally carbon sources by a microorganism into cyanophycin, whereby preferably the nitrogen source comprises nitrogen-containing compounds that are derived from a plant. Suitable microorganisms for use in the processes of the invention include (cyano)bacteria that are naturally capable of synthesising cyanophycin, as well as GMOs that have been engineered to express a (cyano)bacterial cyanophycin synthetase. Such suitable microorganisms capable of producing cyanophycin that may be applied in the processes of the invention are described in more detail herein below.

The nitrogen-containing compounds that are derived from a plant comprised in the nitrogen source preferably comprise organic nitrogen containing compounds as may be present in plant material. These will usually include amino acids, peptides, nucleotides, nucleosides and the like. Preferably in a process according to the invention at least 20, 40, 50, 60, 70, 80, 90, 95 or 99% of the nitrogen atoms in the nitrogen source are present in nitrogen-containing compounds that are derived from a plant. More preferably all nitrogen-containing compounds in the nitrogen source are derived from a plant. Nevertheless, processes in which organic nitrogen-containing compounds from non-plant sources are present or in which inorganic nitrogen compound such as ammonia, nitrate or nitrite are present are not excluded from the present invention.

In a preferred process of the invention at least 10, 20, 30, 40, 50, 60, 70, 80, 90, or 100% of the nitrogen fed is incorporated into the cyanophycin (on a molar basis).

In a preferred process according to the invention the plant-derived nitrogen containing compound(s) that are use as nitrogen source are derived from plants that are capable of nitrogen fixation. The skilled person is aware that such plants do not actually fix the nitrogen themselves but that they obtain nitrogen in a symbiotic relationship with bacteria such as Rhizobium, associated with leguminous plants, and Spirillum lipoferum, associated with cereal grasses. Preferred plants from which nitrogen containing compounds are derived are thus cereal grasses (see below herein) and plants of the family Leguminosae. Plants of the legume family for use in the present invention include e.g. soybeans, alfalfa (Medicago, preferably Medicago sativa), clover, cowpeas, lupines, lucerne, peanuts and other legumes such as e.g. (bean) plants from the genera Phaseolus, Pisum, Vigna, Lens, Cicer, and Soja.

In a preferred process according to the invention the nitrogen source comprises a process stream containing nitrogen, preferably in the form of plant derived nitrogen-containing compounds whereby the process stream is in the processing of agricultural crops. A preferred process according to the invention is a process for valorising a process stream obtained from the processing of agricultural crops, whereby the process stream contains nitrogen, preferably in the form of plant derived nitrogen-containing compounds. Particularly suitable nitrogen-containing process streams for use in the present invention are process streams that are obtained as a by-product in the processing of an agricultural crop. The process stream may e.g. be obtained as in by-product in a process of producing carbohydrate, lipids, oils, fats, proteins, fibers and the like from the agricultural crop. Examples of such processes for producing carbohydrate are e.g. processes in which starch, sugars or cellulose are produced from crop plants. Preferred crop plants for commercial starch production include e.g. potato, corn, cassava. In the processing of starch from corn steep water and/or corn steep liquor are e.g. obtained as nitrogen-containing process streams and similarly, in the processing of starch from potato or cassava the fruit juice that is obtained after rasping and extraction of starch is suitable as a nitrogen-containing process stream. Other crop plants for commercial starch production from which nitrogen-containing process streams may be obtained in process for producing carbohydrate (starch) include amaranth, arrowroot, banana, barley, millet, oat, rice, rye, sago, sorghum, sweet potato, wheat and yam. In addition, similar plant juices are obtainable after extraction processes from grasses (grass juice) such as common grass (Gramineae), from Lolium spp. or from legumes such as Medicago spp.

A highly suitable source of nitrogen-containing compounds that are derived from a plant for use in a process of the invention is concentrated potato fruit juice as is e.g. commercially available from AVEBE (Veendam, The Netherlands) under the name Protamylasse™ (see Example 1). Protamylasse™ and similar concentrated fruit juices and steep waters from starch-crops contain a wide variety of nutrients that can be used as carbon and nitrogen sources for microbial growth and as precursors for biosynthesis of cyanophycin. Beside these nutrients, fruit juices and steep waters from starch-crops like Protamylasse™ contains all possible minerals required for microbial growth. Therefore, in a preferred embodiment of the invention, the nitrogen-containing process stream is used as a complex medium for microbial production of cyanophycin in a process according to the invention without the addition of further nutrients. The application of process streams like Protamylasse™ as N- (and C-) source or as complex medium for cyanophycin production not only renders the biotechnological process economically feasible, because of the low costs of such by-product streams as compared to other complex media or mineral salts media, it is also environmentally friendly, as it provides a useful application for this waste stream. Currently, Protamylasse™ is discarded by epandage as a low cost fertiliser due to its high contents of potassium and phosphate.

The optimal concentration of the plant-derived nitrogen source and/or the nitrogen-containing process streams, whether used as nitrogen source or complex medium, for growth and cyanophycin production by the microorganism may be determined experimentally for each combination of production organism, fermentation conditions and source of nitrogen. This is however routine experimentation for the skilled person (see Example 1). Similarly, further nutrients like additional carbon sources or minerals may be added for optimal growth and cyanophycin production as deemed appropriate by the skilled person. On the other hand, if components are present in the Protamylasse™ that might negatively interfere with cell growth and/or cyanophycin productivity, such as its relatively high potassium concentration (about 13.7% w/w), the use of halotolerant native or genetically modified microorganisms may be considered (see e.g. Boch et al., 1997, for the production of a potassium-tolerant E. coli strain).

Alternatively, juices and extracts from cereal grass, normal grass (Lolium sp.) and/or (green leaves of) legumes such as e.g. alfalfa (Medicago sativa) and lucerne (Medicago sativa L. subsp. sativa) may be applied as the plant-derived nitrogen source and/or complex medium for microbial cyanophycin production in the processes of the invention. Cereal grass is the young green plant which will grow to produce the cereal grain. These young grasses are, in their chemical and nutritional composition, very different from the mature seed grains. Suitable soil, moisture, and temperature conditions for growth of cereal grasses are known to the skilled person. Cereal grass is preferably harvested for production of juice as the plants approach the brief, but critical, jointing stage when the nutrients levels in the plant reach their peak values. Juices and extracts from cereal and normal grasses and legumes may be produced by methods known in the art (see e.g. WO 00/40788 disclosing in general methods for obtaining and separating juice and fiber streams from plant materials). Juices and extracts for use in the present invention may be produced from green leaves of cereals like wheat, barley, rye, rye grass and oats, from normal grass (Lolium sp.), and from legumes such as alfalfa, lucerne or other legumes as indicated herein above and/or mixtures of these cereals, grasses and legumes. The juice may be used as such or it may be dehydrated for storage and to be reconstituted prior to use.

Preferably, in the processes of the invention the plant-derived nitrogen-containing compounds comprise amino acids. More preferably the plant-derived nitrogen-containing compounds comprise at least one or more of amino acids that are present in the cyanophycin to be produced: aspartic acid and either arginine or lysine or optionally other amino acids to be included in the cyanophycin (see below herein). Although these amino acids can be produced by industrial scale fermentations (Leugtenberger, 1996) amounting for L-arginine: 60-100 g per L (Chibata et al., 1983), for L-aspartic acid: 166 g per L (Terasawa et al., 1985) and for L-lysine: 120 g per L (Oh et al., 1990), the production costs would be too high for bulk cyanophycin production. Most preferably, the plant-derived nitrogen-containing compounds comprise at least arginine as de novo biosynthesis by the microorganism of this amino acid requires more energy than e.g. aspartic acid. Preferably, the plant-derived nitrogen-containing compounds comprise at least 0.2, 0.5, 1.0, 2.0, 5.0, 10, 20 or 50% of arginine (w/w on dry weight basis). Preferably, the plant-derived nitrogen-containing compounds comprise at least 0.2, 0.5, 1.0, 2.0, 5.0, 10, 20 or 50% of aspartic acid (w/w on dry weight basis). In a preferred aspect, the plant-derived nitrogen-containing compounds are from a plant that has been genetically modified for increased levels of one or more of aspartic acid, arginine, lysine and another amino acid that is to substitute for arginine in the cyanophycin produced. Preferably the plant is genetically modified for increased metabolic flux towards or a reduced consumption of one or more of these amino acids. Modified plants that exhibit increased pools of one or more of these amino acids may be obtained e.g. by UV mutagenesis and/or anti-sense RNA or RNAi knock down to reduce or inactivate the expression of one or more genes that encode for an enzyme selected from the group consisting of: L-arginine amidinohydrolase (arginase, EC 3.5.3.1, thus also preventing the formation of urea), L-arginine iminohydrolase (EC 3.5.3.6) L-arginine, NADPH:oxygen oxidoreductase (nitric-oxide-forming, EC 1.14.13.39), L-aspartate:2-oxoglutarate aminotransferase (EC 2.6.1.1), L-amino-acid:oxygen oxidoreductase (deaminating, EC 1.4.3.2), L-aspartate:oxygen oxidoreductase (deaminating, EC 1.4.3.16), L-asparagine amidohydrolase (EC 3.5.1.1), L-glutamine(L-asparagine) amidohydrolase (EC 3.5.1.38), L-aspartate:ammonia ligase (AMP-forming, EC 6.3.1.1) and the L-aspartate:L-glutamine amido-ligase (AMP-forming, EC 6.3.5.4) genes (for aspartic acid), L-lysine:oxygen 2-oxidoreductase (deaminating, EC 1.4.3.14) and L-lysine, NADPH:oxygen oxidoreductase (6-hydroxylating, EC 1.14.13.59).

A preferred process according to the invention includes a cyanophycin accumulation phase, in which an inhibitor of ribosomal protein synthesis is added, such as e.g. chloramphenicol and aminoglycosides for prokaryotes or cycloheximide and puromycin for eukaryotes.

In a process according to the invention the microorganism that is used to convert the nitrogen source into cyanophycin, may be a bacterium that naturally produces cyanophycin. A suitable bacteria that naturally produces cyanophycin may be selected from Aphanocapsa, Synechococcus, Synechocystis, Anabaena, Acinetobacter, Spirulina and Desulfitobacterium. The bacterium that naturally produces cyanophycin may be genetically modified for increased production of cyanophycin.

Alternatively, the process according to the invention may be a process wherein the cyanophycin producing microorganism is a microorganism that does not naturally produce cyanophycin but that has been genetically modified to contain an expressible cyanophycin synthetase (cphA) gene and, optionally an expressible cyanophycin depolymerase (cphB) gene and/or an expressible cyanophycin hydrolase (cphE) gene and/or an expressible cyanophycinase (cphI) gene. Suitable examples of these genes are given in Appendix 1 herein. The microorganism that does not naturally produce cyanophycin but that has been genetically modified to produce cyanophycin may be a bacterium, a yeast, a fungus or an alga. The bacterium preferably is a Gram-negative bacterium like e.g. E. coli, Pseudomonas putida and Ralstonia eutropha. Preferably the bacterium is a polyhydroxyalkanoate-negative (PHA) mutant, as e.g. described in Voss et al. (2004). Suitable expression constructs for expression of the cphA, cphB, cphE and/or cphI genes in bacteria are generally known in the art (see e.g. Frey et al., 2002; Voss et al., 2004).

In the process of the invention, the cyanophycin may also be produced by a eukaryotic microorganism that has been genetically modified to contain an expressible cyanophycin synthetase (cphA) gene and, optionally an expressible cyanophycin depolymerase (cphB) gene and/or an expressible cyanophycin hydrolase (cphE) gene and/or an expressible cyanophycinase (cphI) gene. Suitable eukaryotic microorganism for this purpose include yeasts such as e.g. Saccharomyces, Pichia, Kluyveromyces, Hansenula, Candida and Cryptococcus, and filamentous fungi such as e.g. Aspergillus, Penicillium, Rhizopus, and Trichoderma. Suitable expression constructs for expression of the cphA, cphB, cphE and/or cphI genes in yeast and filamentous fungi are generally known in the art (see e.g. Fleer et al., 1991; WO 90/14423; EP-A-0 481 008; EP-A-0 635 574; U.S. Pat. No. 6,265,186).

Transformation of host cells with the nucleic acid constructs for expression of the cphA, cphB, cphE and/or cphI genes in bacteria, yeasts, fungi or algae may be carried out by methods well known in the art. Such methods are e.g. known from standard handbooks, such as Sambrook and Russel (2001) “Molecular Cloning: A Laboratory Manual (3^{rd} edition), Cold Spring Harbor Laboratory, Cold Spring Harbor Laboratory Press, or F. Ausubel et al, eds., “Current protocols in molecular biology”, Green Publishing and Wiley Interscience, New York (1987). Methods for transformation and genetic modification of fungal host cells are known from e.g. EP-A-0 635 574, WO 98/46772, WO 99/60102 and WO 00/37671.

In the nucleic acid constructs for expression of a cphA, cphB, cphE and/or cphI gene in a bacterium, a yeast or a fungus, the genes are operably linked to a promoter that is capable of driven transcription of the gene in the bacterial, yeast, fungal or algal host cell. Suitable promoters for use in bacterial host cells include the native promoters of the cphA, cphB, cphB and/or cphI genes. The genes may be transcribed from constitutive promoters or from environmentally or chemically inducible promoters as are available in the art for bacteria, yeasts and filamentous fungi. Such promoters will usually be heterologous to the cphA, cphB, cphE and/or cphI genes. Typical constitutive and inducible promoters include, but are not limited to, the constitutive Lambda PL promoter with or without the temperature sensitive c1857 repressor or the inducible lacZ promoter for E. coli, the constitutive Ps2 promoter and the Psl inducible by toluene for P. putida, the PBAD promoter inducible with 0.01% L-arabinose for R. eutropha. Similarly, constitutive promoters (GPD, TKL, PGK, GAP, MRP7, TDH3, etc.) as well as inducible promoters (AOX1, XYL1, CUP1, GAL 1, ACIA. etc.) have been described in the art for yeasts and filamentous fungi.

To improve expression of the cphA, cphB, cphE and/or cphI genes in bacteria, yeasts, fungi or algae, the coding sequences of these genes may be adapted to optimise its codon usage to that of the microbial host cell (that does not naturally express a the cphA, cphB cphE and/or gene cphI). The adaptiveness of a coding nucleotide sequence to the codon usage of the host cell may be expressed as codon adaptation index (CAI). The codon adaptation index is herein defined as a measurement of the relative adaptiveness of the codon usage of a gene towards the codon usage of highly expressed genes. The relative adaptiveness (w) of each codon is the ratio of the usage of each codon, to that of the most abundant codon for the same amino acid. The CAI index is defined as the geometric mean of these relative adaptiveness values. Non-synonymous codons and termination codons (dependent on genetic code) are excluded. CAI values range from 0 to 1, with higher values indicating a higher proportion of the most abundant codons (see Sharp and Li, 1987; Jansen et al., 2003). An adapted coding nucleotide sequence for use in the present invention preferably has a CAI of at least 0.2, 0.3, 0.4, 0.5, 0.6 or 0.7.

In a preferred process according to the invention, the microorganism does not only express a cyanophycin synthetase (cphA) gene but also a cyanophycinase (cphI), depolymerase (cphB) and/or hydrolase (cphE). Elbahloul et al. (2005) have found that inactivation of the cyanophycinase gene in Acinetobacter resulted in significantly less cyanophycin accumulation than the wild type. The cyanophycinase releases primer molecules from initially synthesized cyanophycin and higher concentrations of primer molecules produce higher rates of cyanophycin accumulation. The cyanophycin depolymerase (cphB) and/or hydrolase (cphE) have a similar effect.

Depending on the type cyanophycin synthetase expressed in the microorganism, and on the amino acid available in the microorganism the cyanophycin produced may have different amino acid than arginine attached to the poly-aspartic acid backbone. E.g. the cyanobacterial cyanophycin synthetases characterized so far also accept lysine as alternative substrate to arginine. In contrast, the enzyme from A. calcoaceticus strain ADP1 does not accept lysine as alternative to arginine (Krehenbrink and Steinbuchel, 2004). The present invention thus include processes wherein cyanophycins are produced wherein arginine is partially or completely substituted for by lysine or one or more other amino acids. Moreover, given these differences in substrate specificity between the cyanophycin synthetases of Desulfitobacterium and Acinetobacter, it seems reasonable to assume that in vitro mutagenesis and/or gene shuffling may result in aberrant active sites that favour the incorporation of alternative amino acids.

In a preferred process according to the invention, the microorganism has been genetically modified for increased metabolic flux towards or a reduced consumption of one or more of aspartic acid, arginine, lysine and another amino acid that is to substitute for arginine in the cyanophycin produced. Mutant microorganisms that exhibit increased pools of one or more of these amino acids for obtaining enhanced cyanophycin levels may be obtained e.g. by UV mutagenesis and/or insertional mutagenesis whereby one or more genes are inactivated that encode for an enzyme selected from the group consisting of: L-arginine amidinohydrolase (arginase, EC 3.5.3.1, thus also preventing the formation of urea), L-arginine iminohydrolase (EC 3.5.3.6) L-arginine, NADPH:oxygen oxidoreductase (nitric-oxide-forming, EC 1.14.13.39), L-aspartate:2-oxoglutarate aminotransferase (EC 2.6.1.1), L-amino-acid:oxygen oxidoreductase (deaminating, EC 1.4.3.2), L-aspartate:oxygen oxidoreductase (deaminating, EC 1.4.3.16), L-asparagine amidohydrolase (EC 3.5.1.1), L-glutamine(L-asparagine) amidohydrolase (EC 3.5.1.38), L-aspartate:ammonia ligase (AMP-forming, EC 6.3.1.1) and the L-aspartate:L-glutamine amido-ligase (AMP-forming, EC 6.3.5.4) genes (for aspartic acid), L-lysine:oxygen 2-oxidoreductase (deaminating, EC 1.4.3.14) and L-lysine, NADPH:oxygen oxidoreductase (6-hydroxylating, EC 1.14.13.59).

A preferred process of the invention is a process comprising two phases, wherein the first phase comprises accumulation (growth) of biomass of the microorganism, and the second phase comprises accumulation of the cyanophycin. Preferably in the first phase little or no cyanphycin is produced, e.g. less than 40, 30, 20, 10, or 5% of the total cyanophycin produced in the process (weight %). More preferably, in the second phase little or no microbial biomass is produced, e.g. less than 40, 30, 20, 10, or 5% of the total microbial biomass produced in the process (weight %). There are various means available in the art to effect such two-stage processes. E.g the genes encoding enzymes required for or involved in the synthesis of cyanophycin may be controlled by inducible promoters that are switched on at the start of the second phase. Such promoters may be switched on by a change in the culture conditions, e.g. depletion of a nutrient, the addition of an inductor or a shift in temperature (using e.g. a temperature sensitive repressor). Alternatively a promoter may be used that automatically switch on at a certain growth stage, e.g. at or near the stationary phase of the culture.

In a preferred process of the invention the cyanophycin is recovered from the microorganism and optionally purified. Recovery of cyanophycin from the microbial biomass will usually involve disruption of the cells of the microorganism and separation of the cyanophycin from other cell components by e.g. differential centrifugation (see e.g. U.S. Pat. No. 6,180,752). Disruption of the cells of the microorganism may involve the use of an homogeniser, such as e.g. a Cyclone, as are know in the art. Isolation of cyanophycin may also achieved by a simple acid extraction procedure which allows large-scale purification of cyanophycin from whole cells as described by Frey et al. (2002) as follows: First CGP (cyanophycin) is solubilized at about pH 1 in 0.1 N HCl and extracted from biomass without destruction of the structural integrity of the cells. Cyanophycin is recovered from the cells with high yield (about 97%) in two sequential extraction steps. Repeated cycles (2-3 times) of precipitation and solubilization of cyanophycin by i) neutralization of the acidic solution (pH adjustment of extract to 7) and washing of the cyanophycin precipitate with water, and ii) re-solubilization of cyanophycin in HCl lead to isolation of highly purified cyanophycin. Addition of small amounts of other low priced hydrolytic enzymes like lipases or proteases which might accelerate the purification process without contributing significantly to purification costs is in principle possible because cyanophycin is highly resistant to all hydrolytic enzymes tested so far which are not cyanophycinases (reviewed by Obst and Steinbüchel, 2004).

The simple extraction method and spontaneous sedimentation of cyanophycin in aqueous suspensions makes the application of other low price methods like for example filtration of cyanophycin suspensions through commonly employed industrial sieves applicable. Optimization of such a purification method contributes to further reduction of cyanophycin production costs by shortening the time required for sedimentation. The cyanophycin may optionally be further purified by methods known in the art.

In another aspect the invention relates to a process for the production of (a) a cyanophycin with a low arginine content relative to the poly-aspartic acid content with percentages of 10, 20, 30, 40, 50 60, 70, 80, 90, 95, 99% (of the polymer consisting of aspartic acid on a molar basis); and (b) free arginine. The process comprises the step of hydrolysing cyanophycin under mild acidic or mild basic conditions. Preferably, the cyanophycin is obtained in a process for producing cyanophycin as defined herein above. Arginine elimination can take place both with acid and with base (see Example 3.1). If an acidic hydrolysis is carried out, preferably stoichiometric amounts of acid in relation to the incorporated arginine are used because the acid is trapped as arginine salt. It is possible to employ as acid all inorganic acids such as, for example, hydrochloric acid, sulfuric acids, phosphoric acids and lower (i.e. C₁-C₅) fatty acids and other lower (i.e. C₁-C₅) organic acids. The hydrolytic cleavage may also be performed under pressure using carbonic acid or CO₂. Depending on the concentration of the acid employed and on the reaction conditions, depolymerization by hydrolytic cleavage of the polyaspartate chain may take place, in addition to the arginine elimination. However, if depolymerization is not desired it can be minimized by suitable choice of the reaction conditions, such as dilute acid, moderate reaction times, temperatures not exceeding 100° C., preferably between 75-90° C. However, preferably hydrolytic release of arginine from cyanophycin is be carried out under basic conditions, because the polyaspartate chain is more stable under these conditions. The reaction is carried out at a pH above 8.5, preferably 9-12, and at temperatures between 20° C. and 150° C., preferably 50° C.-120° C. Suitable as base for the alkaline hydrolysis are all metal hydroxides or carbonates which make pH values>8.5 possible in aqueous medium. Alkali metal and alkaline earth metal hydroxides are preferred. After the alkaline hydrolysis, the reaction product may be removed by filtration from the unreacted cyanophycin and the alkali-insoluble arginine. The process further comprise the step of recovery, and may further comprise the step of further purification of the cyanophycin with a low arginine content, the poly-aspartic acid and the arginine by differential precipitation, filtration or centrifugation or a combination thereof. A cyanophycin with low arginine content is herein understood to mean a cyanophycin wherein at least 20, 40, 50, 60, 70, 80, 90, 95% of the arginine residues are removed from the poly-aspartic acid backbone.

In a further aspect the invention relates to a process for the production of (1) free aspartic acid and (2) free arginine. The process comprises the step of hydrolysing cyanophycin under more drastic acidic or alkaline conditions. Preferably, the cyanophycin is obtained in a process for producing cyanophycin as defined herein above. Acidic conditions that release arginine and lysine from the poly-aspartic acid backbone and that hydrolyse the poly-aspartic acid backbone into free aspartic acid include e.g. a temperature of 95-100° C.; concentrated strong base or strong acid such as e.g. 2-6 N NaOH or 2-6 N HCl. Differential precipitation of arginine/lysine and aspartic acid derived from the completely hydrolyzed cyanophycin may be performed subsequently under acidic conditions (pH<2) to yield aspartic acid crystals, followed by alkaline conditions (pH>9) for precipitation of arginine, or vice versa. Excess water may subsequently be evaporated.

In a yet another aspect the invention relates to a process for the production of ornithine and urea preferably from arginine. The arginine is preferably obtained from cyanophycin in processes that are defined above. Arginine may be transformed into ornithine and urea by the enzyme arginase, which cleaves arginine into ornithine and urea. This process forms a part of the urea cycle in nature and is catalysed with arginase, an enzyme that is e.g. present in the liver (Bach, 1939; Albanese et al., 1945; Gingras, 1953; Sugino et al., 1952). More recent studies provide a more detailed description of the enzymatic mechanism (Ash, 2001; Ash, 2004; Xie et al., 2004). In addition to the enzymatic approach, arginine may also be hydrolysed using clays such as montmorillonite (Ikeda et al., 1984). The process may further comprise recovery and, optionally further purification of the ornithine and urea.

In a yet another aspect the invention relates to a process for the production of 1,4-butanediamine from ornithine. The ornithine is preferably obtained from arginine in processes that are defined above. Ornithine can undergo a decarboxylation yielding 1,4-butanediamine. This decarboxylation process can be considered in generic terms the decarboxylation of an α-amino acid. This type of reaction is well documented in the open literature for enzymatic decarboxylation using e.g. the enzyme ornithine decarboxylase (EC 4.1.1.17) (Kaye, 1984; www.brenda.uni-koeln.de). In addition a number of chemical methods for the decarboxylation α-amino acids using ketones (Hashimoto et al., 1986), temperature (Li and Brill, 2003) and photolysis (Takano) have been reported. The production of 1,4-butanediamine coupled with urea, as products from arginine isolated from cyanophycin makes an interesting and industrially applicable set of products. The process may further comprise recovery and, optionally further purification of the 1,4-butanediamine.

In a yet another aspect the invention relates to a process for the modification of cyanophycin side chains using enzymatic, physical and chemical methods. Preferably, the cyanophycin is obtained in a process for producing cyanophycin as defined herein above. Such modifications of the cyanophycin molecule may lead to change in the chemical functionality, architecture and consequently the chemical and physical properties. In cyanophycin the arginine (and/or lysine) side chains may undergo chemical reactions such as esterification at the free —COOH group and, in the case where lysine side chains are present, formation of amide bonds by reaction at the ε-NH₂ position. Alternatively the arginine (and/or lysine) side chains may undergo enzymatic modification. A number of experimental procedures have been reported in the literature for the treatment of arginine with arginase (www.brenda.uni-koeln.de), however similar procedures using cyanophycin resulted in no reaction (Simon 1987; Obst and Steinbüchel, 2004). Cyanophycin may however be treated with arginine iminohydrolase (EC 3.5.3.6), peptidyl-arginine deiminase (EC 3.5.3.15) and/or nitric oxide synthetase (EC 1.14.13.39), which results in the arginine side chains in cyanophycin being modified to citrulline side chains. In turn these citrulline side chains may be modified to ornithine side chains using enzyme citrulline phosphorylase (EC 2.1.3.3) resulting in the formation of L-ornithine side chains. The process is thus a process for producing an ornithine functionalised cyanophycin or rather an ornithine functionalised poly(aspartic acid). The process may further comprise recovery and, optionally further purification of the ornithine functionalised-cyanophycin or -poly(aspartic acid). Further modifications of the arginine side chains of cyanophycin may be effected by treating a cyanophycin with the following enzymes together with the required co-substrates or co-factors as indicated:
argininosuccinate lyase (EC 4.3.2.1) together with fumarate to produce (N-L-arginino)succinate functionalised poly(aspartic acid).
arginine kinase (EC 2.7.3.3) together with ATP to produce N-phospho-L-arginine functionalised poly(aspartic acid).
arginine-glycine transamidinase (EC 2.1.4.1) with glycine to produce L-ornithine functionalised poly(aspartic acid).
L-arginine carboxy-lyase (EC 4.1.1.19) to produce agmatine functionalised poly(aspartic acid).

In related aspects the invention relates to ornithine functionalised poly(aspartic acid), (N-L-arginino)succinate functionalised poly(aspartic acid), N-phospho-L-arginine functionalised poly(aspartic acid) and agmatine functionalised poly(aspartic acid) or compositions comprising one or more of these functionalised poly(aspartic acids). The degree of functionalisation in the functionalised poly(aspartic acids), as well as in the compositions in which they are comprised or in the above processes in which they are produced may be varied by the skilled person by varying the reaction conditions as may be determined by routine experimentation. The degree of functionalisation in the functionalised poly(aspartic acids), compositions and processes may thus be such that at least, or alternatively, less than 10, 20, 30, 40, 50, 60, 70, 80, 90, 95, 99% of the arginine residues present in the cyanophycin starting material are functionalised. Preferably all arginine in the cyanophycin is functionalised, in as far as this is detectable. From www.brenda.uni-koeln.de 120 hits with 29 families of enzymes (from different organisms) that interact with arginine or any of its derivatives were retrieved. Some of the enzymes listed above were able to modify the terminal group of the arginine side chain (—NH—C(═N)—NH₂) group rather than the α—NH group used to connect the arginine side chain to the poly(aspartic acid) backbone.

In a yet another aspect the invention relates to a process for the modification of the lysine contained in the side chains of the cyanophycin molecule using enzymatic methods. Preferably, the cyanophycin is obtained in a process for producing cyanophycin as defined herein above. Modifications of the lysine side chains of cyanophycin may be effected by treating a cyanophycin with the following enzymes together with the required co-substrates or co-factors as indicated:
Lysine N6-hydroxylase (EC 1.14.13.59) to produce N6-hydroxy-L-lysine functionalised poly(aspartic acid).
D-lysine 5,6-aminomutase (EC 5.4.3.4) to produce 2,5-diaminohexanoate functionalised poly(aspartic acid).
Lysine-2-oxoglutarate reductase (EC 1.5.1.7) with 2-oxoglutarate+NADH+H+ to produce N6-(L-1,3-dicarboxypropyl) functionalised poly(aspartic acid).
Lysine-ketoglutarate reductase (EC 1.5.1.8) with 2-oxoglutarate+NADPH+H+) to produce N6-(L-1,3-dicarboxypropyl) functionalised poly(aspartic acid).
Carboxy-lyases (EC 4.1.1.18) to produce poly(aspartic acid) functionalised with pentanediamine.
Lysine oxygenase (EC 1.13.122) to produce poly(aspartic acid) functionalised with 5-aminopentanamide.
Lysine acetyltransferase (EC 2.3.1.32) with acetyl phosphate to produce poly(aspartic acid) functionalised with N6-acetyl-L-lysine.

In related aspects the invention relates to N6-hydroxy-L-lysine, 2,5-diaminohexanoate, N6-(L-1,3-dicarboxypropyl), pentanediamine, 5-aminopentanamide or N6-acetyl-L-lysine functionalised poly(aspartic acid), or compositions comprising one or more of these functionalised poly(aspartic acids). The degree of functionalisation in the functionalised poly(aspartic acids), as well as in the compositions in which they are comprised or in the above processes in which they are produced may be varied by the skilled person by varying the reaction conditions as may be determined by routine experimentation. The degree of functionalisation in the functionalised poly(aspartic acids), compositions and processes may thus be such that at least, or alternatively, less than 10, 20, 30, 40, 50, 60, 70, 80, 90, 95, 99% of the lysine residues present in the cyanophycin starting material are functionalised. Preferably all lysine in the cyanophycin is functionalised, in as far as this is detectable.

In yet another aspect the invention relates to a process in which any amino acid other than arginin or lysine are incorporated into the side chain and further functionalised using enzymatic, physical or chemical modifications (as described above).

In a yet another aspect the invention relates to a process in which cyanophycin, preferably obtained in a process for producing cyanophycin as defined herein above, initially undergoes an enzymatic (or physical or chemical) modification (as described above), followed by a chemical or physical modification. Those chemical modifications, carried out post enzymatic modification, may lead to the formation of esters and amides.

In a yet another aspect the invention relates to a process in which cyanophycin, preferably obtained in a process for producing cyanophycin as defined herein above, and cyanophycin which has undergone enzymatic (or chemical) modification(s) (as described above), may be used to prepare blends with either other cyanophycin derived modified polymers or other polymers of natural or synthetic origin.

In a yet another aspect the invention relates to a process to the production of one or more of maleic acid, fumaric acid, succinic acid and 1,4-butanediol. The maleic acid, fumaric acid, succinic acid and 1,4-butanediol are preferably produced from aspartic acid obtained in a process as defined above. It is known that under high temperature reactions aspartic acid more readily undergoes α-deamination than α-decarboxylation. The present process therefore comprises the step of thermally treating aspartic acid to produce maleic acid and/or fumaric acid and ammonia, under the conditions described by Sohn and Ho (1995) or Sato (2004). In subsequent steps the maleic acid (or fumaric acid) may be reduced to form succinic acid, which in a further step may be reduced to form 1,4-butanediol. The process may further comprise recovery and, optionally further purification of the maleic and/or fumaric acid, succinic acid and/or 1,4-butanediol.

In a yet another aspect the invention relates to a process to the production of n-alkyl amino alcohols, preferably amino propanol. The n-alkyl amino alcohols are preferably produced from aspartic acid obtained in a process as defined above. The production of n-alkyl amino alcohols, preferably amino propanol, from aspartic acid, requires the removal of the α-carboxylic acid group, followed by reduction of the carboxylic acid functionality to a hydroxyl group. This may be carried out by employing reaction conditions, which effect decarboxylation at the α-position as opposed to deamination. This may be done photochemically (physically) (Takano and Kaneko) or by enzymatic means using enzyme aspartate 1-decarboxylase (EC 4.1.1.1.1). The reduction of the carboxylic acid functionality to a hydroxyl group may be carried out using methods described in literature for such a chemical transformation. The process thus comprising the step of decarboxylation aspartic acid at the α-position whereby the aspartic acid is obtained in a process defined above and whereby the aspartic acid is decarboxylated photochemically or by treatment with enzymes, followed by reduction of the carboxylic acid functionality to a hydroxyl group, and may further comprise recovery and, optionally further purification of the n-alkyl amino alcohol.

In a yet another aspect the invention relates to a process to the production of acrylonitrile. Acrylonitrile is preferably produced from aspartic acid obtained in a process as defined above. The initial step involves α-decarboxylation of aspartic acid (described above) followed by reduction of the carboxylic acid group and finally dehydration to an alkene bond, coupled with the dehydrogenation of the primary amine to a nitrile, produces acrylonitrile. The process may further comprise recovery and, optionally further purification of the acrylonitrile.

In the enzymatic modifications of the cyanophycins of the invention, as described herein above, preferably one or more of the modifying enzymes that are used are active at an acidic or alkaline pH, preferably at a pH at which the cyanophycins of the invention are water soluble. Preferably, the enzymes are active at a pH above 8.0, 8.5 or 9.0, or at a pH below 4.0, 3.0 or 2.0. More preferably the enzymes have an acidic or alkaline pH optimum. Preferably the pH optimum of the enzymes is above pH 7.0, more preferably at pH 8.0, 8.5 or 9.0, or the pH optimum of the enzymes is below pH 7.0, more preferably at pH 6.0, 5.0, 4.0, 3.0 or 2.0. Such enzymes may e.g. be obtained from alkaliphilic or acidophilic micro-organisms, respectively.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### EXAMPLES

### Example 1

### Production of Cyanophycin by Escherichia coli and Other Genera Using Protamylasse as a Substrate

Escherichia coli strain DH1, which contains the vector pMa/c5-914::cphA (pMa/c5-914 carrying 2.6-kb PCR product from Synechocystis sp. strain PCC6803 genomic DNA harboring cphA), is grown on Protamylasse™ (concentrated fruit juice from the potato starch productions) as is obtainable from AVEBE, Veendam, the Netherlands. Protamylasse™ contains a wide variety of nutrients that can be used as carbon and nitrogen sources for growth of E. coli and as precursors for biosynthesis of cyanophycin. Beside these nutrients, Protamylasse™ contains all possible minerals required for microbial normal growth. E. coli DH1(pMa/c5-914::cphA) is cultured using different concentrations of Protamylasse™ in order to determine the optimal concentration for growth and cyanophycin production. Cells of E. coli DH1(pMa/c5-914::cphA) are able to synthesize 25.6±3.9 and 26.8±1.2% (wt/wt) cyanophycin when cells grown in 5% and 6% (v/v) Protamylasse™ with initial pH of 7 and supplemented with 100 μg/ml ampicillin, respectively. The cells are inoculated from a pre-culture previously grown in Protamylasse™ at 30° C., then the main cultures are incubated for 44 h at 37° C. in order to induce cyanophycin synthesis. Higher concentrations of Protamylasse, however, are not suitable for cyanophycin synthesis in E. coli DH1(pMa/c5-914::cphA). Changing the initial pH value shows that the optimum pH of 7.5 results in a cyanophycin content of 27.2±3.3% (wt/wt) of cell dry matter. Cells of E. coli DH1(pMa/c5-914::cphA) cultivated on Protamylasse™ as complex medium are shown to have higher cyanophycin contents than cells cultivated on TB complex medium, which produce about 24% (wt/wt) of cells dry matter. Cyanophycin is extracted easily by stirring the cells overnight in water at a pH value of about one using the advantage that cyanophycin is soluble at low pH. The cyanophycin polymer is then precipitated and separated by neutralizing the acidic solution. The cyanophycin is composed of aspartic acid, arginine and lysine, the latter comprise only up to 10% of the total amino acids contents. The fermentation studies using 30 L stirred fermenter and applying diluted Protamylasse™ 6% (v/v) with initial pH value of 7.5 as complex medium supplemented with 100 μg/ml ampicillin results in cyanophycin contents of 26.2±2.3% (wt/wt) of cells dry matter. The molecular weight of cyanophycin as determined by SDS-PAGE is about 30 KDa, which is similar to that produced using TB complex medium. The application of Protamylasse™ as complex medium for cyanophycin production making the biotechnological process not only economically feasible, because the costs of Protamylasse™ are much lower in comparison to other complex media or mineral salts media, it is also environmentally friendly, because it provides a useful application of this waste stream and residual compounds. Comparison of standard growth media (Mineral Salts medium with or without Cas amino acids, or Terrific Broth) with 6 vol % Protamylasse™ for the 30 L lab-scale cyanophycin production process using the same E. coli DH1(pMa/c5-914::cphA) strain shows that Protamylasse™ results in the highest cyanophycin yield, despite its lower cell dry matter yield (Appendix 4, Table 1). Similar results were observed in experiments using E. coli DH1(pMa/c5-914::cphA) in addition to Acinetobacter calcoaceticus strain ADP1 (Example 2) and different grass juice-based media.

### Example 2

### Production of Cyanophycin by Acinetobacter Calcoaceticus ADP1 Using Grass Liquid Concentrate as a Substrate

Acinetobacter calcoaceticus strain ADP1 was shown recently to contain active cyanophycin synthetase (Krehenbrink et al. 2002) and is able to synthesize high cyanophycin contents using arginine as sole carbon source (Elbahloul et al. 2005). As known, the application of arginine as sole source of carbon is not economically feasible. Therefore, an alternative substrate which is rich in arginine must be applied in order to minimize the costs and sustain the high productivity of the cells for cyanophycin. Specific types of grass juice concentrates (the composition of grass juice is indicated in Appendix 3) can be applied as such media using either the wild type strain of Acinetobacter calcoaceticus or other mutants, which are able to overproduce arginine and aspartic acid. Acinetobacter calcoaceticus ADP1 is able to produce more than 40% of cell dry matter of cyanophycin when cultivated on arginine. This high productivity was the maximum amount of cyanophycin ever reported. Cyanophycin is extracted by the simple acid extraction method. In addition, cyanophycin composed only of aspartic acid and arginine will be produced because the cyanophycin synthetase of A. calcoaceticus ADP1 exhibits a high substrate specificity and does not incorporate for example lysine. The molecular weight of cyanophycin produced by A. calcoaceticus ADP1 is in the range of 25 to 28 kDa, and is therefore smaller than that produced by recombinant strains.

### Example 3

### Production of Arginine from Cyanophycin Under Mild or Drastic Conditions

### 3.1 Mild Alkaline or Acidic Hydrolysis of Cyanophycin

About 1 g of cyanophycin extracted from biomass (Examples 1 or 2) is suspended in 10-20 ml of water and are stirred for 15 h in the presence of 50-200 mg of NaOH, KOH) or diluted inorganic or organic acids like for example HCl, H2SO4, H3PO4, formic acid, acetic acid, propionic acid or butyric acid at temperatures between 75-90° C. The pH value of the resulting mixture is adjusted to approximately 7 and water content is subsequently reduced by evaporation of the solvent. Cyanophycin with low arginine content and free arginine are successively precipitated according to their different solubility products (see below). Precipitated polymer and arginine crystals are successively removed from the mixture by filtration and precipitates are subsequently dried.

### 3.2 Hydrolysis of Cyanophycin Under Drastic Conditions

The maximum theoretical yield of free arginine from 1 g cyanophycin is about 0.6 g and about 0.46 g of free aspartic acid is yielded if total hydrolysis occurs under more drastic conditions (95-100° C.; concentrated base or acid; for example 2-6 N HCl). Fractionated precipitation of arginine and aspartic acid derived from totally hydrolyzed cyanophycin is performed under acidic conditions (pH<2) to yield aspartic acid crystals, under alkaline conditions (pH>9) for arginine by reduction of the water content by evaporation.

A comparable adjustment of the pH value is alternatively applied to precipitate arginine and cyanophycin with reduced arginine content which are produced under mild hydrolysis conditions (see above).

### REFERENCES

1. Aboulmagd E, Oppermann-Sanio F B, Steinbüchel A. Molecular characterization of the Cyanophycin synthetase of Synechocystis sp. PCC6308. Arch. Microbiol. 174 (2000) 297-306.
2. Aboulmagd E, Oppermann-Sanio F B, Steinbüchel A. Purification of the Synechocystis sp. strain PCC6308 Cyanophycin synthetase and its characterization with respect to substrate and primer specificity. Appl. Environ. Microbiol. 67 (2001) 2176-2182.
3. Aboulmagd E, Voβ I, Oppermann-Sanio F B, Steinbüchel A. Heterologous expression of Cyanophycin synthetase and Cyanophycin synthesis the industrial relevant bacteria Corynebacterium glutamicum and Ralstonia eutropha and in Pseudomonas putida. BioMacromolecules 2 (2001) 1338-1342.
4. Albanese A A, Irby V. et al. J. Biological Chem. 160 (1945) 25-30
5. Allen M M, Weathers P. J. Bacteriol. 141 (1980) 959.
6. Ash D E. Abstracts of Papers, 22^{nd} ACS National Meeting, Chicago, August 2001 (2001).
7. Ash D E. J. Nutrition, 134 (2004) 2760S-2764S.
8. Bach S J. Biochemical Journal 33 (1939) 1833-1844.
9. Berg H, Ziegler K, Piotukh K, Baier K, Lockau W. Eur. J. Biochem. 267 (2000) 5561.
10. Boch J, Nau-Wagner G, Kneip S, Bremer E. Glycine betaine aldehyde dehydrogenase from Bacillus subtilis: Characterization of an enzyme required for the synthesis of the osmoprotectant glycine betaine. Archives of Microbiology. 168 (1997) 282-289.
11. Borzi A. Malpighia 1 (1887) 74.
12. Cibata I, Kisumi M, Tagaki T. Jpn Patent (Kokai, 1983) 58-9692.
13. Elbahloul Y, Krehenbrink M, Reichelt R, Steinbüchel A. Physiological conditions for high Cyanophycin content in biomass of Acinetobacter calcoaceticus strain ADP1. Appl. Environ. Microbiol. 71 (2005) 858-866.
14. Fleer et al., Biotechnology 9 (1991) 968-975.
15. François I E J A et al. Processing in Arabidopsis thaliana of a heterologous polyprotein resulting in differential targeting of the individual plant defensins. Plant Sci. 166 (2004) 113-121.
16. Frey K M, Oppermann-Sanio F B, Schmidt H, Steinbuchel A. Technical scale production of Cyanophycin with recombinant strains of E. coli. Appl. Environ. Microbiol. 68 (2002) 3377-3384.
17. Füser G, Steinbüchel A. Investigations on the solubility behavior of Cyanophycin—solubility of Cyanophycin in solutions of simple inorganic salts. Biomacromolecules (2005), revised version submitted.
18. Gingras R. Laval Medical, 18 (1953) 724-744
19. Hai T, Oppermann-Sanio F B, Steinbüchel A. Purification and characterization of Cyanophycin and Cyanophycin synthetase from the thermophilic Synechococcus sp. MA19. FEMS Microbiol. Lett. 181 (1999) 229-236.
20. Hai T, Ahlers H, Steinbüchel A. Axenic cultivation of anoxygenic phototrophic bacteria, cyanobacteria and microalgae in a new closed tubular glass photobioreactor. Appl. Microbiol. Biotechnol. 53 (2000) 383-389.
21. Hai T, Oppermann-Sanio F B, Steinbüchel A. Molecular characterization of a thermostable Cyanophycin synthetase from the thermophilic cyanobacterium Synechococcus sp. strain MA19 and in vitro synthesis of Cyanophycin and related polyamides. Appl. Environ. Microbiol. 68 (2002) 93-101.
22. Halpin C et al. Enabling technologies for manipulating multiple genes on complex pathways. Plant Mol. Biol. 47 (2001) 295-310.
23. Hashimoto M, Eda Y, Osanai Y, Iwai T. Chem. Lett (1986) 893-896.
24. Hejazi M, Piotukh K, Mattow J, Deutzmann R, Volkmer-Engerts R, Lockau W. Biochem. J. 364 (2002) 129.
25. Ikeda T, Yasunaga T. J. Phys. Chem. 88 (1984) 1253-1257.
26. Jansen et al., 2003, Nucleic Acids Res. 31 (2003) 2242-2251
27. Kaye A M. Cell Biochemistry and Function 2 (1984) 2, and references therein.
28. Krehenbrink M, Oppermann-Sanio F B, Steinbüchel A. Evaluation of non-cyanobacterial genome sequences for occurrence of Cyanophycin synthetase homologous genes and cloning of an active Cyanophycin synthetase from Acinetobacter sp. DSM 587. Arch. Microbiol. 177 (2002) 371-380.
29. Krehenbrink M, Steinbüchel A. Partial purification and characterization of a non-cyanobacterial Cyanophycin synthetase from Acinetobacter calcoaceticus strain ADP1 with regard to substrate specificity, substrate affinity and binding to Cyanophycin. Microbiology 150 (2004) 2599-2608.
30. Leuchtenberg W. 1996. Amino acids—Technical Production and use (Chapter 14a). In: Biotechnology, Products of primary metabolism (vol. 6). H.-J. Rehm and G. Reed (eds.). VCH Verlagsgeselschaft mbh, Weinheim, Germany. Pp 466-502.
31. Li J, Brill T B. J. Phys. Chem. 107 (2003) 5987-5992.
32. Liotenberg S, Campbell D, Rippka R, Hourmard J, Tandeau de Marsac N. Microbiology 142 (1996) 611.
33. Mackerras A H, De Chazal N M, Smith G D. J. Gen. Microbiol. 136 (1990) 2057.
34. Nakano K, Rischke M, Sato S, Märkl H. Influence of acetic acid on the growth of Escherichia coli K12 during high-cell-density cultivation in a dialysis reactor. Appl. Microbiol. Biotechnol. 48 (1997) 597-601.
35. Obst M, Oppermann-Sanio F B, Steinbüchel A. Isolation of Cyanophycin degrading bacteria, cloning and characterization of an extracellular Cyanophycinase gene (cphE) from Pseudomonas anguilliseptica strain BI—The cphE gene from P. anguilliseptica BI encodes a Cyanophycin-hydrolyzing enzyme. J. Biol. Chem. 277 (2002) 25096-25105.
36. Obst M, Sallam A, Luftmann H, Steinbüchel A. Isolation and characterization of Gram-positive Cyanophycin degrading bacteria—kinetic studies on Cyanophycinase activity in aerobic bacteria. Biomacromolecules 5 (2004) 153-161.
37. Obst M, Steinbüchel A. Microbial degradation of poly(amino acid)s. Biomacromolecules 5 (2004) 1166-1176.
38. Oh J W, Kim S J, Cho Y J, Park N H, Lee L H. Nouveau microorganisme capable de produire de la L-lysine at procéde dé fermentation l'utilisant pour produire de la L-lysine. French Patent Appl. (1990) 2 645 172.
39. Oppermann-Sanio F B, Steinbüchel A. Occurrence, functions and biosynthesis of polyamides in microorganisms and biotechnological production. Naturwissenschaften 89 (2002) 11-22.
40. Oppermann-Sanio F B, Steinbüchel A. 2002 Cyanophycin. In: Fahnestock, S. R. and A. Steinbüchel (eds.) Biopolymers, Vol. 7, 1^{st} Edition, Wiley-VCH, Weinheim, pp. 83-106.
41. Oppermann-Sanio F B, Steinbüchel A. 2005. Cyanophycin. In: Steinbüchel, A., and Y. Doi (Eds.) Biotechnology of Biopolymers—From Synthesis to Patents. Volume 2, pp. 767-790, WILEY-VCH, Weinheim.
42. Richter R, Hejazi M, Kraft R, Ziegler K, Lockau W. Eur J. Biochem. 263 (1999) 163-169.
43. Sato, N.; Quitain, A. T.; Kang, K.; Daimon, H.; Fujie, K. Ind. Eng. Chem. Res. 43(13) (2004) 3217.
44. Sharp and Li. Nucleic Acids Research 15 (1987) 1281-1295
45. Simon R D. Proc. Natl. Acad. Sci. U.S.A. 68 (1971) 265.
46. Simon R D, Weathers P. J. Bacteriol. 114 (1973) 1213.
47. Simon R D, Weathers P. Biochim. Biophys. Acta 420 (1976) 165.
48. Simon R D. Arch. Microbiol. 92 (1973) 115.
49. Simon R D. Inclusion bodies in the cyanobacteria: Cyanophycin, polyphosphate, polyhedral bodies. In The cyanobacteria; Fay, P., van Baalen, C., Eds.; Elsevier: Amsterdam, 1987; pp 199-225.
50. Simon R D. Biochim. Biophys. Acta 422 (1976) 407.
51. Sohn M, Ho C-T. J. Agri. Food Chem. 43 (1995) 3001.
52. Sugino K, Odo K et al. Nippon Kagaku Kaishi (1921-47), Pure Chem. Sect. 73 (1952) 214-216
53. Takano Y, Kaneko T. et al. ISSN 1343-8832, Terra Scientific Publishing Co. (Abstract information).
54. Terasawa M, Yukawa H, Takayama Y. Production of L-aspartic acid from Brevibacterium by the cell reusing process. Process Biochem 20 (1985) 124-128.
55. Voss I, Diniz S C, Aboulmagd E, Steinbüchel A. Identification of the Anabaena sp. strain PCC7120 Cyanophycin synthetase as suitable enzyme for production of Cyanophycin in Gram-negative bacteria like Pseudomonas putida and Ralstonia eutropha. Biomacromolecules 5 (2004) 1588-1595.
56. Wetzel T et al. Size and sequence variability of the Arabis mosaic virus protein 2A. Arch. Virol. 147 (2002) 1643-1653.
57. www.brenda.bc.uni-koeln.de
58. Xie X Y, Wang C X, Wang A Y. J. Thermal Analysis and Calorimetry 76 (2004) 275-284.
59. Ziegler K, Deutzmann R, Lockau W. Z. Naturforsch. C 57 (2002) 522.
60. Ziegler K, Diener A, Herpin C, Richter R, Deutzmann R, Lockau W. Eur. J. Biochem. 254 (1998) 154.

### APPENDIX 1

Cyanophycin Synthetase (cphA) and Depolymerase (cphB, cphE and cphI) Gene Sequences that can be Used for PCR Primer Design and/or Expression Vector Construction

Annotated Cyanophycin synthetase (cphA) and depolymerase/hydrolase/cyanophycinase (cphB, cphE and cphI) gene sequences from:

Bordetella pertussis Tohama, (BP1740GeneID: 2665843);
Bordetella bronchiseptica RB50 (BB3584GeneID: 2662715);
Bordetella pertussis Tohama I (BP1739GeneID: 2665842);
Craterostigma plantagineum homeodomain leucine zipper protein CPHB-7 (CPHB-7) mRNA, complete cds, gi|18034444|gb|AF443623.1|[18034444];
Craterostigma plantagineum homeodomain leucine zipper protein CPHB-6 (CPHB-6) mRNA, complete cds, gi|18034442|gb|AF4436221.1|[18034442];
Craterostigma plantagineum homeodomain leucine zipper protein CPHB-5 (CPHB-5) mRNA, complete cds, gi|18034440|gb|AF443621.1|[18034440];
Craterostigma plantagineum homeodomain leucine zipper protein CPHB-4 (CPHB-4) mRNA, complete cds, gi|18034438|gb|AF443620.1|[18034438];
Craterostigma plantagineum homeodomain leucine zipper protein CPHB-3 (CPHB-3) mRNA, partial cds, gi|18034436|gb|AF443619.1|[18034436];
Clostridium perfringens str. 13, complete genome, gi|18308982|ref|NC_{—}003366.1|[18308982];
Clostridium perfringens str. 13 (CPE2213 GeneID: 990537); Clostridium perfringens str. 13 DNA, complete genome, gi|47118322|dbj|BA000016.3|[47118322];
Clostridium tetani E88 (CTC00282GeneID: 1059860);
Francisella tularensis subsp. tularensis Schu 4 (FTT1130cGeneID: 3191888);
Francisella tularensis subsp. tularensis Schu 4, complete genome, gi|56707187|ref|NC_{—}006570.1|[56707187];
Francisella tularensis subsp. tularensis complete genome, gi|56603679|emb|AJ749949.1|[56603679];
Gloeobacter violaceus PCC 7421 (gvip562GeneID: 2602729);
Idiomarina loihiensis L2TR, complete genome, gi|56459112|ref|NC_{—}006512.1|[56459112];
Idiomarina loihiensis L2TR, complete genome, gi|56178122|gb|AE017340.1|[56178122];
Nitrosomonas europaea ATCC 19718 (NE0923GeneID: 1081864);
Nitrosomonas europaea ATCC 19718 (NE0922GeneID: 1081863);
Nostoc sp. PCC 7120 (all3879GeneID: 1107477);
Nostoc sp. PCC 7120 (alr0573GeneID: 1104169);
Nostoc sp. PCC 7120, complete genome, gi|17227497|ref|NC_{—}003272.1[17227497];
Nostoc sp. PCC 7120 DNA, complete genome, gi|47118302|dbj|BA000019.2|[47118302];
Oryza sativa (japonica cultivar-group) genomic DNA, chromosome 9, BAC clone: OJ1596_{—C}06, gi|51536099|dbj|AP005575.3|[51536099]
Pseudomonas anguilliseptica strain BI 16S ribosomal RNA gene, complete sequence gi|21744718|gb|AF439803.1|[21744718];
Pseudomonas anguilliseptica extracellular Cyanophycinase (cphE) gene, complete cds, gi|21744226|gb|AY065671.1|[21744226];
Synechococcus sp. MA19 Cyanophycinase (cphB) and Cyanophycin synthetase (cphA) genes, complete cds, gi|18033114|gb|AF329282.1|AF329282[18033114];
Synechococcus elongatus cphB gene for Cyanophycinase and cphA gene for cyanphycin synthetase, gi|10047070|emb|AJ288949.1 SEL288949[10047070];
Synechocystis PCC6308 cph gene cluster, complete sequence, gi|13516213|gb|AF220099.2|AF220099[13516213];
Thermoanaerobacter tengcongensis MB4, complete genome, gi|20806542|ref|NC_{—}003869.1|[20806542];
Thermoanaerobacter tengcongensis MB4, section 243 of 244 of the complete genome gi|20517791|gb|AE013216.1|[20517791];
Thermosynechococcus elongatus BP-1(tlr2170GeneID: 1011138);
Tolypothrix sp. PCC 7601 phytochrome-like protein (cphB) and response regulator (rcpB) genes, complete cds, gi|8642522|gb|AF309560.1|[18642522];
Yersinia pestis KIM, complete genome, gi|22123922|ref|NC_{—}004088.1|[22123922];
Yersinia pestis KIM section 221 of 415 of the complete genome, gi|21959014|gb|AE013821.1|[21959014].

### APPENDIX 2

Codon Usage Table of Synechocystis sp. PCC 6803 [gbbct]: 3766 CDS's (1207436 Codons) http://www.kazusa.or.jp/codon/
fields: [triplet] [frequency: per thousand] ([number])

### UUU 29.3 (35370) UCU 9.0 (10838) UAU 17.3 (20912) UGU 6.3 (7634) UUC 10.7 (12936) UCC 15.9 (19239) UAC 12.0 (14519) UGC 3.9 (4692) UUA 26.1 (31487) UCA 4.3 (5139) UAA 1.4 (1681) UGA 0.6 (760) UUG 29.0 (34970) UCG 4.0 (4875) UAG 1.1 (1325) UGG 15.5 (18673)

CUU 10.1 (12210) CCU 10.0 (12031) CAU 11.6 (14022) CGU 10.4 (12533) CUC 14.0 (16885) CCC 24.6 (29646) CAC 7.3 (8772) CGC 12.2 (14752) CUA 13.9 (16764) CCA 8.0 (9643) CAA 33.7 (40730) CGA 5.3 (6449) CUG 20.0 (24202) CCG 8.3 (9978) CAG 21.1 (25463) CGG 13.4 (16233)
AUU 40.0 (48321) ACU 13.8 (16694) AAU 25.5 (30749) AGU 14.9 (18011) AUC 18.0 (21718) ACC 26.2 (31630) AAC 15.1 (18268) AGC 10.2 (12365) AUA 4.8 (5837) ACA 6.9 (8363) AAA 30.2 (36405) AGA 4.5 (5445) AUG 19.5 (23596) ACG 7.8 (9369) AAG 12.9 (15539) AGG 4.8 (5768)
GUU 16.8 (20320) GCU 20.1 (24319) GAU 32.4 (39150) GGU 20.1 (24229) GUC 11.2 (13528) GCC 37.7 (45537) GAC 17.9 (21633) GGC 22.4 (27050) GUA 10.6 (12771) GCA 10.8 (13004) GAA 44.7 (54017) GGA 12.9 (15522) GUG 28.1 (33936) GCG 15.2 (18335) GAG 16.0 (19314) GGG 17.7 (21330)
Coding GC 48.32% 1st letter GC 55.85% 2nd letter GC 39.76% 3rd letter GC 49.37%

### APPENDIX 3

### Composition of Grass or Lucerne Juice.

In addition to amino acids (see below) grass or lucerne juice contains the following components: vitamins A, B, C, E and K, calcium, chlorophyll, iron, lecithin, magnesium, pantothenic acid, phosphorus, potassium, trace elements and protein (up to 30%).

The wheat and Barley cereal grass promotional literature of the 1950s claimed that cereal grasses contain every nutrient known to be required by humans except vitamin D, which is made in the skin. Contemporary laboratory analyses show that a wide variety of nutrients are contained in dehydrated cereal grasses. Some of these nutrients are quite concentrated; others are present only in small amounts. These nutrients are combined by nature to provide a uniquely potent food.

The following Table summarizes the levels of known nutrients contained in the cereal grasses. The nutrient concentrations depend on the growing conditions and the growth stage at which the cereal grasses are harvested, rather than on the type (barley, rye, or wheat) of cereal grass analyzed (http://www.naturalways.com/grass.htm).

APPENDIX 3
In house determination of the composition of
grass juice and lucerne juice (molasse)

| | Grass juice (g · kg⁻¹) | Lucerne juice (g · kg⁻¹) |
|---|---|---|
| dry substance | 586 | 613 |
| pH | 4.9 | 5.8 |
| Potassium | 47.3 | 105.3 |
| Sodium | 0.97 | 4.44 |
| calcium | 4.25 | 2.35 |
| magnesium | 1.87 | 4.7 |
| ammonia | 0.35 | 1.41 |
| citric acid | 3.4 | 51.2 |
| malic acid | 29.7 | 14.5 |
| lactic acid | 39.9 | 7.8 |
| acetic acid | 0 | 13.1 |
| phosphate | 11.29 | 12.72 |
| sulphate | 12.6 | 17.7 |
| nitrate | 1.2 | 0.3 |
| chloride | 7.5 | 33.7 |
| raw protein (N × 6.25) | 55 | 152 |
| aspartic acid | 5.3 | 31.5 |
| asparagine | — | — |
| glutamic acid | 4.2 | 25 |
| isoleucine | 1.2 | 1.6 |
| leucine | 1.8 | 1.1 |
| methionine | 0.5 | 1.5 |
| cystine | 0.4 | 1.2 |
| phenylalanine | 1.3 | 2.5 |
| tyrosine | 0.7 | 1.1 |
| threonine | 1.5 | 2 |
| tryptophane | 0.2 | 0.1 |
| valine | 2.1 | 3.9 |
| alanine | 3.7 | 8.3 |
| glycine | 1.7 | 2.6 |
| proline | 2.2 | 3.4 |
| serine | 1.4 | 2.2 |
| g-aminobutyric acid | 2.5 | 8.3 |
| ornithine | 0.2 | 0.5 |
| lysine | 1 | 2.8 |
| arginine | 0.6 | 3.1 |
| histidine | 0.3 | 0.4 |
| total amino acids | 32.8 | 103.1 |
| acid amino acids | 9.5 | 56.5 |
| neutral amino acids | 21.4 | 40.3 |
| basic amino acids | 1.9 | 6.3 |

### APPENDIX 4

Frey et al. (2002) have used the recombinant E. coli DH1(pMa/c5-914::cphA) containing the Synechocystis sp. PCC6803 cphA gene for fermentative (30 L) cyanophycin production in different media, as follows: standard Mineral Salts medium without or with Casein amino acids (Nakano et al., 1997) and TB (Terrific Broth, Opperman-Sanio et al., 1999). In a comparison with 30 L fermentation experiments performed with Protamylasse™ results are obtained as shown in Table 1, demonstrating that the 6% (v/v) crude preparation of Protamylasse™ (that contains 60% of dry matter) results in the highest efficiency of cyanophycin (CGP) synthesis despite its lowest biomass yield.

TABLE 1
Process data on cyanophycin synthesis by recombinant
E. coli DH1(pMa/c5-914::cphA)

| | C-source(s)/ | N-source(s)/ | | CGP |
|---|---|---|---|---|
| | concentration | concentration¹ | CDM | content |
| Medium | (g · L⁻¹) | (g · L⁻¹) | (g · L⁻¹) | wt % |
| MS² + gluc. 20 | glyc. 40 | 1.0 | 13.0 | traces |
| | | ([NH4]2SO4 | | |
| | | 0.1 (NH4Cl) | | |
| MS/CasAA + | glyc. 20 | 10 | 13.8 | 12 |
| gluc. 20 | | | | |
| TB | glyc. 3.2 | 36² (Trypt.; | 6.7-8.3 | 21-24 |
| | | YE) | | |
| Protamylasse ™³ | sugars: 7.2 | AA: 5.53 | 5.0 | 26-27 |
| | org. acids: | | | |
| | 6.8 | | | |

¹pH control by HCl and/or NaOH, not NH₃
²MS, Mineral Salts medium, components (per L), Nakano et al., 1997); Terrific Broth, components (per L): Tryptone, 12.0 g, Yeast Extract (YE), 24.0 g, K₂HPO₄, 9.4 g, KH₂PO₄, 2.2 g, Glycerol, 4 ml (Sigma catalogue 2005); CasAA, Tryptone, YE have a mean content of 0.5 g.L−1 of arg (as in ProtamylasseTM (The Oxoid Manual, 6th edn, 1990).
³Protamylasse ™ contains per 1000 g dry matter: total amino acids: 257 g; arg: 12.9 g; asx: 91.4 g; total sugars (including fructose, glucose, saccharose): 200 g, organic acids (including citric, malic, oxalic, acetic, lactic acid): 190 g, ash: 317 g, biotin: 0.05 mg; Ca-pantothenate: 64 mg; folic acid: 2 mg; nicotinic acid: 280 mg; Vit B1: <0.1 mg; Vit B2: 7 mg; Vit. B6: 31 mg.
CDM: cell dry matter;
CGP: cyanophycin granule polypeptide;
n.d.: no data,
AA: all amino acids.

1. A process for producing cyanophycin, wherein the process comprises conversion of a nitrogen source, and optionally a carbon source, by a microorganism into cyanophycin and wherein the nitrogen source comprises nitrogen-containing compounds that are derived from a plant.

2. A process according to claim 1, wherein at least 50% of the nitrogen atoms in the nitrogen source are present in nitrogen-containing compounds that are derived from a plant, preferably all nitrogen-containing compounds in the nitrogen source are derived from a plant.

3. A process according to claim 1, wherein the plant is in symbiosis with a nitrogen-fixing bacterium, preferably the plant is a leguminous plant or a cereal grass.

4. A process according to claim 1, wherein the nitrogen source comprises a process stream containing nitrogen, the process stream being obtained in the processing of agricultural crops.

5. A process according to claim 4, wherein the process stream is obtained as a by-product in a process of producing carbohydrate, lipid, oil, fat, protein, or fiber from the agricultural crop.

6. A process according to claim 5, wherein the process stream comprises potato or cassava fruit juice, or corn steep water or steep liquor.

7. A process according to claim 4, wherein the process stream is a cereal grass juice, or a alfalfa or a lucerne (Medicago) juice.

8. A process according to claim 1, wherein the least 20% of the nitrogen fed is incorporated in the cyanophycin (on a molar basis).

9. A process according to claim 1, wherein the plant-derived nitrogen-containing compounds comprises one or more of the amino acids that are present in the cyanophycin that is produced in the process.

10. A process according to claim 1, wherein the microorganism is a bacterium that naturally produces cyanophycin, preferably selected from Aphanocapsa, Synechococcus, Synechocystis, Anabaena, Spirulina, Acinetobacter and Desufitobacterium.

11. A process according to claim 1, wherein the microorganism is a microorganism that does not naturally produce cyanophycin and wherein the microorganism has been genetically modified to contain an expressible cyanophycin synthetase (cphA) gene and, optionally an expressible cyanophycin depolymerase (cphB) gene and/or an expressible cyanophycin hydrolase (cphE) gene and/or an expressible cyanophycinase (cphI) gene.

12. A process for producing:
(a) cyanophycin with a low arginine content and/or poly-aspartic acid; and,
(b) free arginine;
the process comprising the step of hydrolysing cyanophycin as obtained in a process according to claim 1, under mild acidic or mild basic conditions and recovery and, optionally further purification of the cyanophycin with a low arginine content, the poly-aspartic acid and the arginine.

13. A process for producing:
(a) aspartic acid; and/or,
(b) arginine;
the process comprising the steps of hydrolysing cyanophycin as obtained in a process according to claim 1, under strong acidic or strong basic conditions; and recovery and, optionally further purification of the aspartic acid and the arginine.

14. A process for producing ornithine and urea, the process comprising the steps of enzymatic or (thermo)chemical hydrolysis of arginine as obtained in a process according to claim 12; and recovery and, optionally further purification of the ornithine and urea.

15. A process for producing 1,4-butanediamine, the process comprising the steps of enzymatic or chemical decarboxylation of ornithine as obtained in a process according to claim 14; and recovery and, optionally further purification of the 1,4-butanediamine.

16. A process for producing maleic acid and/or fumaric acid, the process comprising the steps of thermally treating aspartic acid obtained in a process according to claim 13; and recovery and, optionally further purification of the maleic and/or fumaric acid.

17. A process for producing succinic acid, the process comprising the steps of reducing maleic and/or fumaric acid obtained in a process according to claim 16; and recovery and, optionally further purification of the succinic acid.

18. A process for producing 1,4-butanediol, the process comprising the steps of reducing succinic acid obtained in a process according to claim 17; and recovery and, optionally further purification of the 1,4-butanediol.

19. A process for producing an n-alkyl amino alcohol, preferably amino propanol, the process comprising the steps of decarboxylation of aspartic acid at the α-position whereby the aspartic acid is obtained in a process according to claim 13 and whereby the aspartic acid is decarboxylated photochemically or by using enzymes followed by reduction of the carboxylic acid group to a hydroxyl group; and recovery and, optionally further purification of the n-alkyl amino alcohol.

20. A process for producing acrylonitrile, the process comprising the steps of:
(a) decarboxylation aspartic acid at the α-position whereby the aspartic acid is obtained in a process according to claim 13 and whereby the aspartic acid is decarboxylated photochemically or by treatment with enzymes;
(b) reduction of the carboxylic acid group followed by dehydration to an alkene bond;
(c) dehydrogenation of the primary amine to a nitrile; and,
(d) recovery and, optionally further purification of the n-alkyl amino alcohol.

21. A process for producing a functionalised poly(aspartic acid), the process comprising the step of treating a cyanophycin as obtained in a process according to claim 1 with:
(a) an arginine iminohydrolase, a peptidyl-arginine deiminase, or a nitric oxide synthetase to obtain a citrulline functionalised poly(aspartic acid), which citrulline functionalised poly(aspartic acid) may optionally be treated with citrulline phosphorylase to obtain an ornithine functionalised poly(aspartic acid); or,
(b) an arginine-glycine transamidinase in the presence of glycine to obtain an ornithine functionalised poly(aspartic acid); or,
(c) an argininosuccinate lyase in the presence of fumarate to obtain a (N^{W}-L-arginino)succinate functionalised poly(aspartic acid); or,
(d) an arginine kinase in the presence of ATP to obtain an N-phospho-L-arginine functionalised poly(aspartic acid);
(e) an L-arginine carboxy-lyase to obtain an agmatine functionalised poly(aspartic acid);
(f) a lysine N6-hydroxylase (EC 1.14.13.59) to produce N6-hydroxy-L-lysine functionalised poly(aspartic acid);
(g) a D-lysine 5,6-aminomutase (EC 5.4.3.4) to produce 2,5-diaminohexanoate functionalised poly(aspartic acid);
(h) a lysine-2-oxoglutarate reductase (EC 1.5.1.7) with 2-oxoglutarate+NADH+H+ to produce N6-(L-1,3-dicarboxypropyl) functionalised poly(aspartic acid);
(i) a lysine-ketoglutarate reductase (EC 1.5.1.8) with 2-oxoglutarate+NADPH+H+) to produce N6-(L-1,3-dicarboxypropyl) functionalised poly(aspartic acid);
(j) a carboxy-lyases (EC 4.1.1.18) to produce poly(aspartic acid) functionalised with pentanediamine;
(k) a lysine oxygenase (EC 1.13.122) to produce poly(aspartic acid) functionalised with 5-aminopentanamide; or,
(l) a lysine acetyltransferase (EC 2.3.1.32) with acetyl phosphate to produce poly(aspartic acid) functionalised with N6-acetyl-L-lysine.

22. A functionalised poly(aspartic acid), wherein at least 50% of the arginine residues is functionalised to citrulline, ornithine, (N^{W}-L-arginino)succinate, N-phospho-L-arginine, agmatine or a combination of one or more of the arginine functionalisations.

23. A functionalised poly(aspartic acid), wherein at least 50% of the lysine residues is functionalised to N6-hydroxy-L-lysine, 2,5-diaminohexanoate, N6-(L-1,3-dicarboxypropyl), pentanediamine, 5-aminopentanamide or N6-acetyl-L-lysine or a combination of one or more of the lysine functionalisations.

24. A process in which the modified side chain functionalities as defined in claim 21 are further chemically, physically and/or enzymatically modified.

25. A process for producing a blend comprising one or more polymers selected from:
(a) a cyanophycin as obtainable in a process according to claim 1;
(b) a cyanophycin with reduced arginine content as obtainable in a process according to claim 12;
(c) a functionalised poly(aspartic acid) as obtainable in a process according to claim 21;
the process comprising the step of mixing at least two polymers selected from (a), (b) and (c) that differ in at least one of molecular weight distribution, arginine content or type or degree of functionalisation.

26. A blend of comprising one or more polymers selected from:
(a) a cyanophycin;
(b) a cyanophycin with reduced arginine content;
(c) a functionalised poly(aspartic acid) as defined in claim 21;
wherein at least two polymers selected from (a), (b) and (c) differ in at least one of molecular weight distribution, arginine content or type or degree of functionalisation.
